(19) United States
(12) Patent Application Publication
(10) Pub. No.: US 2008/0233051 A1
(43) Pub. Date: Sep. 25, 2008

(54) Nanoparticles for two-photon activated photodynamic therapy and imaging

(76) Inventors: Paras N. Prasad, Williamsville, NY (US); Sehoon Kim, Kyung-ki (KR); Tymish Y. Ohulchanskyy, Kenmore, NY (US); Ravindra K. Pandey, Williamsville, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 11/983,845

(22) Filed: Nov. 13, 2007

Related U.S. Application Data
(63) Continuation-in-part of application No. 11/900,334, filed on Sep. 10, 2007.
(60) Provisional application No. 60/843,037, filed on Sep. 8, 2006.

(51) Int. Cl. A61K 9/14; A61K 31/695; A61K 49/00

(52) U.S. Cl. 424/9.6; 424/489; 514/63

(57) ABSTRACT

The present invention provides organically modified silica (ORMOSIL) nanoparticles into which have been incorporated two-photon absorption dye molecules. The two photon absorption dye displays a unique aggregation induced fluorescence enhancement behavior. As a result ORMOSIL nanoparticles with high amounts of the dye can be prepared. These particles can be used for imaging. In one embodiment, the nanoparticles can additionally have incorporated therein a photosensitizer. The photosensitizer can be activated by intraparticle fluorescence resonance energy transfer (FRET) from the dye aggregates resulting in enhanced fluorescence and singlet oxygen generation from photosensitizer under two-photon excitation conditions. Such nanoparticles can be used for photodynamic therapy applications.

This application is a continuation-in-part of U.S. Non-provisional Application No. 11/900,334 filed on Sep. 10, 2007, which in turn claims priority to U.S. Provisional Application No. 60/843,037 filed on Sep. 8, 2006, the disclosures of which is incorporated herein by reference.

This work was supported by funding under Grant No. FA9550-04-1-0158 from the USAF/AFOSR. The Government has certain rights in the invention.

### FIELD OF THE INVENTION

The present invention relates generally to the area of delivery of photosensitive molecules to biological systems and more particularly provides compositions and methods for efficient delivery of two-photon dyes for applications in bioimaging and photodynamic therapy.

### BACKGROUND OF THE INVENTION

Two-photon absorption (TPA) dyes have wide applications, including optical limiting, up-converted lasing, three-dimensional optical data storage, bioimaging and photodynamic therapy (PDT). For biological applications, it is preferred that TPA dyes be water-soluble or dispersable and remain highly fluorescent in aqueous media (Prasad, Introduction to Biophotonics, John Wiley & Sons, New Jersey, 2003). Many of the known TPA dyes, however, are hydrophobic and their fluorescence quantum yields are considerably reduced in water due to self-aggregation induced fluorescence quenching (Birks, Photophysics of Aromatic Molecules, Wiley, London, 1970). To address this problem, polymer nanoparticles have been devised. These nanoparticle carriers enable a stable aqueous dispersion of hydrophobic dyes or drugs, and can be appropriately sized for passive targeting to tumor tissues. However, because of self aggregation induced fluorescence quenching, the amount of dye encapsulated in the nanoparticles is limited resulting in insufficient amounts being delivered to the target tissues.

Photodynamic therapy (PDT) is one of the applications of TPA dyes and is considered to be a promising approach for the treatment for cancer and other diseases. PDT utilizes light-sensitive drugs or photosensitizers which can be preferentially localized in malignant tissues upon systemic administration. The therapeutic effect is initiated by photoexcitation of the localized photosensitizers and the subsequent generation of cytotoxic species, such as singlet oxygen (¹O₂), free radicals or peroxides, which lead to selective and irreversible destruction of the diseased tissues without damaging adjacent healthy ones.

In spite of the advantages of PDT over current treatments including surgery, radiation therapy and chemotherapy, it still has not gained a more general clinical acceptance. One of the reasons is that currently approved photosensitizers absorb in the visible regions of the spectrum below 700 nm, where light penetration into the skin is only a few millimeters, limiting the clinical efficacy to topical ailments. To overcome this problem, TPA-induced excitation of photosensitizers to increase light penetration has been suggested. This enables the use of light in the tissue transparent window (750-1000 nm) and further provides a tool for improving treatment of deeper tumors with enhanced spatial resolution due to a quadratic dependence of TPA on laser intensity.

For efficient two-photon sensitization, one approach is the energy-transferring combination of photosensitizers with TPA dyes, where the photosensitizing unit (energy acceptor) is indirectly excited through fluorescence resonance energy transfer (FRET) from the two-photon absorbing dye unit (energy donor). This approach was realized using chemical assembling of the TPA donors into a dendrimer with a photosensitizer as the central core (Dichtel et al., J. Am. Chem. Soc. 2004, 126, 5380, Oar et al., Chem. Mater. 2005, 17, 2267). Nevertheless, the preparation of pharmaceutical formulations of photosensitizers for parenteral administration poses a challenge in PDT therapy approaches. Since most existing photosensitizers are hydrophobic with poor water solubility, they aggregate easily under physiological condition and thus cannot be simply injected intravenously. Moreover, even with water-soluble photosensitizers, the accumulation selectivity to diseased tissues is not high enough for clinical use. While colloidal carriers, such as oil-dispersions, liposomes, low-density lipoproteins, polymeric micelles, and nanoparticles (Konan et al., J. Photochem. Photobiol., B 2002, 66, 89; van Nostrum, Adv. Drug Deliv. Rev. 2004, 56, 9; Wang et al., M. J. Mater Chem. 2004, 14, 487), offer benefits of hydrophilicity and size, because of self-aggregation induced fluorescence quenching, the amount of TPA dye that can be packed into each carrier is limited. Therefore, the problem of insufficient delivery to the targeted tissues still remains unresolved.

Thus, there is a need in the area of biophotonic applications, particularly such as bioimaging and PDT, to identify efficient delivery systems which will provide efficient delivery of the dyes and/or photosensitizer agents to target tissue in adequate amounts without adversely affecting the functionality of the dyes or photosensitizers.

### SUMMARY OF THE INVENTION

The present invention provides organically modified silica (ORMOSIL) nanoparticles into which have been incorporated two-photon absorption (TPA) dye molecules. The two photon dye displays a unique aggregation induced fluorescence enhancement behavior. As a result ORMOSIL nanoparticles with high amounts of the dye can be prepared. These particles can be used for imaging.

In one embodiment, the nanoparticles can additionally have incorporated therein a photosensitizer. The photosensitizer can be activated by intraparticle fluorescence resonance energy transfer (FRET) from the dye aggregates resulting in enhanced fluorescence and singlet oxygen generation from the photosensitizer under two-photon excitation conditions which does not exhibit aggregation related quenching. Such nanoparticles can be used for photodynamic therapy applications. Suitable TPA dyes are 9,10-Bis[4′-styryl-styryl]anthracenes and a suitable photosensizer is 2-devinyl-2-(1-hexyloxyethyl)pyropheophorbide (HPPH).

### BRIEF DESCRIPTION OF THE DRAWINGS

FIG. 1: Representation of the Reagents and conditions: i) NaO′Bu, methanol, R.T. ii) Pd(OAc)₂, P(o-tolyl)₃, Et₃N, NMP, 80° C. iii) PhCOCl, Et₃N, NMP, R.T. Et₃N: triethylamine; R.T.: room temperature.

FIG. 2: Fluorescence quantum yields (Φf) of BDSA-Bz and BDSB at 5 μM, as a function of water fraction in THF/H₂O mixture. The missing data for BDSA-Bz at 50-60% are owing to bulk precipitation.

FIG. 3: Representative transmission electron microscopic (TEM) images of ORMOSIL nanoparticles incorporating the BDSA aggregates. The BDSA loading [BDSA/VTES] and the mean diameters are (a) 10 wt %, 28.9±8.7 nm, (b) 20 wt %, 27.1±6.7 nm, (c) 30 wt %, 26.7±7.8 nm, and (d) 40 wt %, 27.4±7.8 nm, respectively. In the images, it seems that some parts of particle population have been interconnected to form bigger clusters, which leads to broad size distributions.

FIG. 4: (a) Normalized one-photon excited fluorescence spectra of the BDSA/ORMOSIL composite nanoparticles with varying BDSA compositions (BDSA/[BDSA+VTES] in wt %). (b) One-photon excited quantum yield (open square) and the total fluorescence output by one-photon (solid triangle) and two-photon (solid circle) excitations (each scaled arbitrarily), depending on the BDSA concentration in the composite nanoparticles. The wavelengths for one- and two-photon excitation are 480 and 800 nm, respectively.

FIG. 5. (a) Fluorescence quantum yields of common dyes in ORMOSIL nanoparticles, depending on dye loading. (b) Structures of common dyes examined, and their fluorescence quantum yields in solution, which are plotted in the left part of (a). The quantum yields of nanoparticles were estimated using each dye solution noted in (b) as a reference.

FIG. 6: (a) Two-photon images of HeLa cells incubated with the BDSA/ORMOSIL nanoparticles (20 wt % of BDSA loading [BDSA/VTES]) for 3 hrs. (red: fluorescence, green: transmission). The 800-nm excitation (140 fs, 76 MHz, 10 mW under microscope) was used with spectrally tunable emission filter set to 500-650 nm. (b) Flow cytometry histograms of the BDSA/ORMOSIL nanoparticles in HeLa cells. The BDSA loading is expressed as [BDSA/VTES] in wt %, while keeping the initial feed weights of VTES constant for all samples. The control sample is untreated HeLa cells, the signal from which is autofluorescence. In all cases, 10,000 cells were counted and all data were gated in a similar way.

FIG. 7. Chemical structure of BDSA.

FIG. 8. TEM images of silica nanoparticles entrapping (a) 1.1 wt % HPPH and (b) 1.1 wt % HPPH/20 wt % BDSA.

FIG. 9. Normalized fluorescence spectra of BDSA, entrapped in PMMA films (a: 0.5 wt %, b: 30 wt %) and in water-dispersed silica nanoparticles (c: 20 wt %). Inset: fluorescence quantum yields (Φ_{f}) of BDSA-loaded silica nanoparticles, depending on the loading amount.

FIG. 10. Normalized absorption and fluorescence spectra of HPPH (1.1 wt %) and BDSA (20 wt %) entrapped in water-dispersed silica nanoparticles. The excitation wavelengths are 600 and 480 nm for HPPH and BDSA, respectively.

FIG. 11. One-photon excited fluorescence spectra of the same amount of water-dispersed silica nanoparticles incorporating (a) 20 wt % BDSA, (b) 1.1 wt % HPPH, (c) 1.1 wt % HPPH/20 wt % BDSA. Because of significant overlapping of BDSA and HPPH emission spectra, they were deconvoluted in (c). Curves (d), (e) present the deconvoluted BDSA and HPPH fluorescence spectra, respectively. The excitation wavelength was 425 mn.

FIG. 12. Two-photon excited fluorescence spectra of the same amount of water-dispersed silica nanoparticles incorporating (a) 1.1 wt % HPPH, (b) 1.1 wt % HPPH/10 wt % BDSA, and (c) 1.1 wt % HPPH/20 wt % BDSA. The excitation wavelength is 850 nm.

FIG. 13. BDSA fluorescence decays for nanoparticles doped with (1) 20 wt % BDSA and (2) 20 wt % BDSA/1.1 wt % HPPH. Instrument response function (IRF) is shown unlabeled.

FIG. 14. Emission of singlet oxygen generated in the D₂O dispersion of nanoparticles coincorporating 1.1 wt % HPPH/20 wt % BDSA. The excitation wavelength was 532 nm.

FIG. 15. Photobleaching of 9,10-anthracenedipropionic acid, disodium salt (ADPA) by singlet oxygen generated upon two-photon excitation of the water-dispersed nanoparticles coincorporating 1.1 wt % HPPH/20 wt % BDSA at 850 nm. Inset: ADPA remaining in water dispersions of the nanoparticles encapsulating 1.1 wt % HPPH (square) and 1.1 wt % HPPH/20 wt % BDSA (circle), as a function of time of irradiation with 850 nm.

FIG. 16. Merged transmission (blue) and two-photon excited fluorescence (red) images of HeLa cells, stained with nanoparticles coincorporating 1.1 wt % HPPH/20 wt % BDSA. Inset: Localized two-photon fluorescence spectrum from the cytoplasm of the stained cell. The excitation wavelength is 850 nm.

### DESCRIPTION OF THE INVENTION

The present invention is based on the surprising discovery of a class of TPA dyes which do not exhibit fluorescence quenching upon aggregation. On the contrary, these dyes exhibit aggregation related enhancement in fluorescence. The dyes are 9,10-Bis[4′-styryl-styryl]anthracenes and can be represented by the following Formula 1.

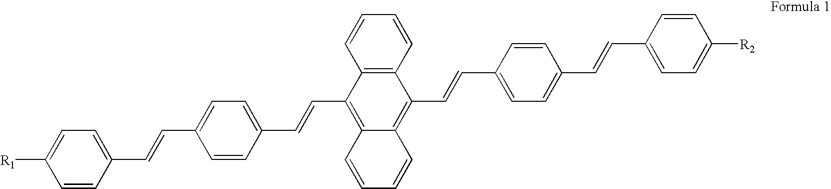

Wherein R¹ and R² can independently be —NX¹X², —OX³, or —SX⁴ and X¹, X², X³ and X⁴ can independently be H, alkyl, hydroxyalkyl, benzoyloxyalkyl, phenyl and naphthyl.

In one embodiment, R¹ and R² are the same. In a particular embodiment, the dye is a 9,10-bis[4′-(4″-aminostyryl)styryl]anthracene. An example of such a dye is shown in FIG. 7 (BDSA).

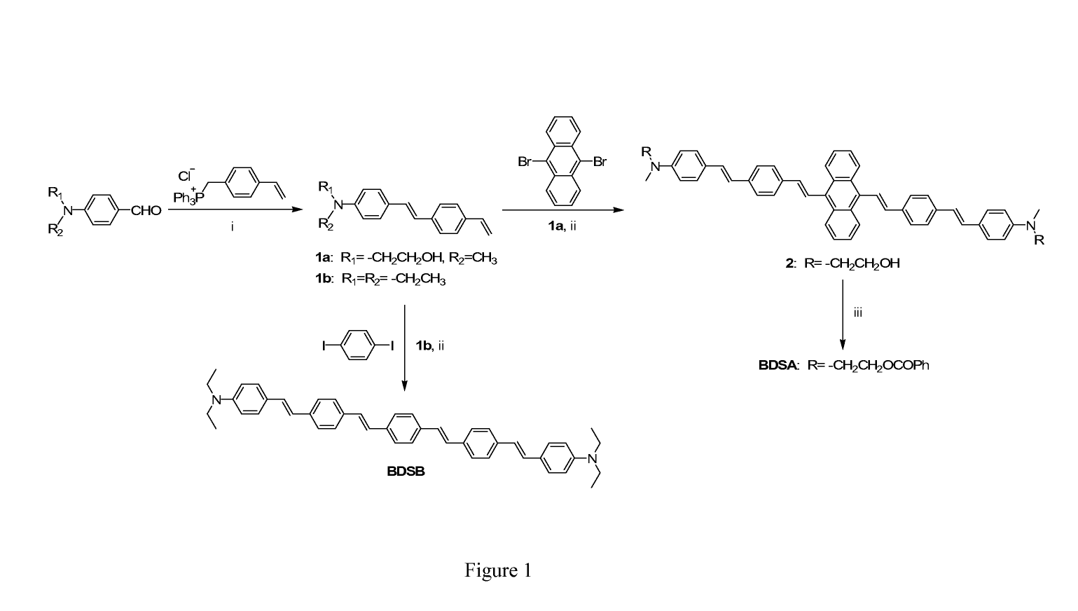

A general synthesis scheme is shown in FIG. 1. Synthetic procedures for 9,10-anthrylene-core BDSA and its 1,4-phenylene-core analogue (BDSB) are depicted in Scheme 1. BDSB is a conventional planar TPA dye which was used for control experiment to ascertain the role of central anthrylene unit in modulating optical properties. The chromophore unit, 2, can be obtained in all-trans form by consecutive reactions of Wittig condensation to give 1a (37% yield) and trans-selective Heck coupling between 1a and 9,10-dibromoanthracene (43% yield). It should be noted that the reaction can be used to give asymmetric products by the coupling, with 9,10-dibromoanthracene, of chromophore units which differ in R-group placement and type. In such a reaction, one would expect to get three different products which could be separated, if necessary, for further use. Two of products would be symmetric, while a third would be asymmetric, bearing one of each of the different chromophore units. For Wittig condensation between N-methyl-N-(2-hydroxyethyl)-4-aminobenzaldehyde and 4-vinylbenzylphosphonium chloride methanol can be used as a solubility limiting solvent, from which only trans isomer of 1a is selectively precipitated during the reaction and, thus, readily isolated by filtration. BDSB was obtained by the same procedure using 4-diethylaminobenzaldehyde for Wittig condensation to give 1b (35% yield) and 1,4-diiodobenzene for Heck coupling with 1b (76% yield). By introducing two benzoyloxy terminal groups in 2, suitable organic solubility and hydrophobicity of the final dye (BDSA) is achieved for the preparation of organic nanoparticle, by the precipitation method (Kasai et al., Handbook of Nanostructured Materials and Nanotechnology, Vol. 5, Academic Press, New York, 2000, Ch. 8). Stable water dispersions of BDSA nanoaggregates can be prepared by a simple precipitation method. THF or N-methyl-2-pyrrolidinone (NMP) is used as a water-miscible solvent for the dye.

The BDSA has one dimensionally elongated π-conjugation for large TPA activity, the quadrupolar framework of which is likely to be distorted severely due to a large internal steric hindrance between the anthrylene center and vinylene moieties substituted at its 9,10-positions. A distorted geometry in the monomeric form limits effective conjugation. In the solid state, however, its conjugation length increases by stacking-induced planarization, and moreover, the partially distorted structure due to the internal steric hindrance, even after stacking, disturbs the close packing, to diminish the intermolecular quenching effects. It was found that the emission of BDSA is unusually quenched in a true solution, but enhanced in bulk solid or nanoaggregated dispersions. More importantly, we also observed notably enhanced large TPA activity in nanoaggregate forms of pure BDSA.

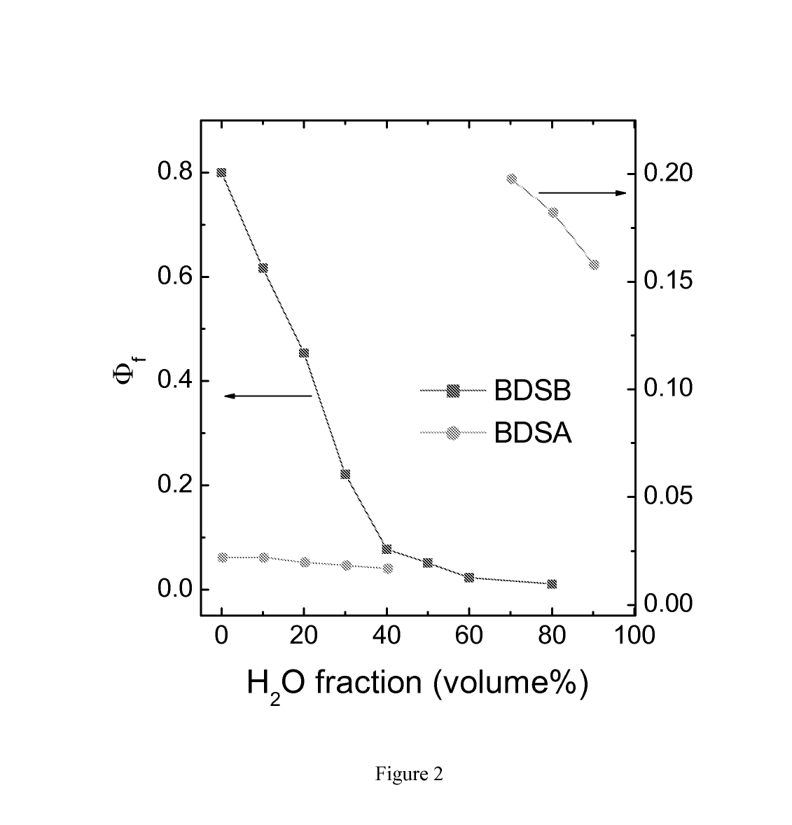

A representative transmission electron microscopic (TEM) image of the obtained nanoaggregates with a diameter of 44±7 nm is shown in FIG. 1a. FIG. 1b shows the unusual fluorescence behavior of BDSA, where the fluorescence was quenched in NMP solution (Φ_{f}˜0.009), but intensified more than 30 times by aggregation. The fluorescence quantum yield (Φ_{f}) of nanoaggregated state varies (0.1˜0.3), depending on the preparation conditions, including solvent, solvent/water ratio, and overall dye concentration, etc. In contrast to fluorescence enhancement, the absorption of BDSA was broadened and showed a hypochromic effect in intensity, by nanoaggregation. No notable sign of H- or J-aggregation was observed, implying that molecular stacking is neither too close nor well ordered within the nanoaggregated structure. The fluorescence quenching in solution can be understood as dominant non-radiative decay, enhanced by free intramolecular torsional motion in solution, as in the case of flexible molecules showing viscosity-dependent fluorescence. Moreover, the weak, broad, and red-shifted emission of the solution, compared to that of the aggregate, implies the existence of large-amplitude relaxation in the excited state, such as twisted intramolecular charge transfer (TICT) which often acts as an intramolecular fluorescence quencher due to the nπ* characteristics. This is supported by our observation of intense and blue-shifted monomer fluorescence from a dilute solid solution of BDSA in PMMA film, where isolated molecules are frozen in the distorted form by rigid matrix, to prevent fluorescence quenching motion. The structural effect on aggregate formation and fluorescence was studied with a specific comparison between BDSA and the 1,4-phenylene analogue, BDSB, by varying the solvent/water ratio. As shown in FIG. 2, aggregate formation is closely correlated with fluorescence alteration. Enhanced fluorescence is seen for BDSA, but not for BDSB.

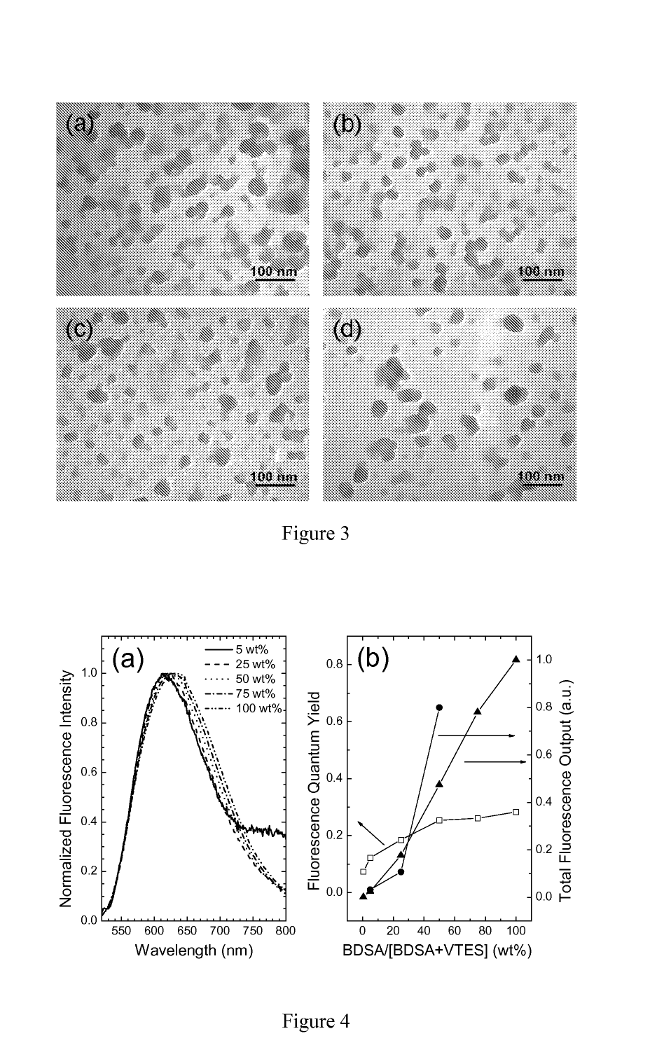

ORMOSIL nanoparticles having incorporated therein the TPA dye can be prepared as follows. Using a homogeneous solution of BDSA and prepolymerized triethoxyvinylsilane (VTES) sol, the BDSA/ORMOSIL composite nanoparticles can be synthesized through transient emulsification of the solution in the nonpolar interior of aqueous Aerosol OT (AOT) micelle and spontaneous co-precipitation via a ‘solvent displacement’ process, i.e., diffusive depletion of the hydrophilic solvent into the aqueous exterior. The particle size was analyzed by transmission electron microscopic (TEM) images, the result of which showed good agreement with the number-averaged size distribution in aqueous suspension, obtained from dynamic light scattering (DLS) (see FIG. 3). Although the composite nanoparticles obtained by this method are polydispersed in size, the average diameters can be controlled to less than 30 nm so as to minimize disturbance of normal cellular physiology. Those skilled in the art will recognize that the size can be controlled by varying the amount of the TPA dye, the precursor, VTES and/or the surfactant, the AOT. Generally, the size of the nanoparticles for the present invention averages from 10 to 100 nm and preferably from 15-30 nm. After removal of the anionic surfactant (AOT) by dialysis against deionized water, all the composite nanoparticles have a negative ζ potential (−25˜−35 mV at pH 7.0), indicating that the dispersions in water are stabilized by a negative surface charge. It is believed that the composite particle surface is composed of the neutral or ionized silanol moiety (≡Si—OH or ≡Si—O⁻) as well as hydrophobic parts (BDSA and vinyl group of VTES). With increasing BDSA loading, the 70 potential becomes more negative, probably due to increased surface hydrophobicity and subsequently promoted preferential adsorption of anions (OH⁻ or residual AOT). After removal of surfactant by dialysis, the nanoparticles are formulated as stable aqueous dispersion. The ORMOSIL nanoparticles have a high concentration of the TPA dye and yet exhibit high fluorescence.

For the present invention, it is preferable to have a BDSA loading range of between 1 wt % to 50 wt %. Enhanced fluorescence was observed for up to 30 wt % and no further significant increase was observed above 40%. Cell viability was not affected up to 50 wt %, but appeared to be affected above 50 wt % as determined by visual inspection of cells in vitro. In a preferred embodiment, the TPA dye can be any integer between and including 5 to 40 wt %, and preferably between 10 to 30 wt %.

For PDT applications, a photosensitizer can be coincorporated into the ORMOSIL nanoparticle along with the TPA dye. In this embodiment, the present invention utilizes intraparticle energy transfer between the TPA dye and the photosensitizer. Thus, the TPA dye and the photosensitizer represents a FRET donor-acceptor pair. The photosensitizer generates cytotoxic singlet oxygen following the fluorescence resonance energy transfer from the two-photon absorption drug. Molecular oxygen as well as reactive oxygen species (singlet oxygen and free radicals) can diffuse through the oxygen-permeable matrix of the ORMOSIL nanoparticle making these nanoparticles useful for PDT. In addition, the ORMOSIL particles are rigid enough to preserve the initially loaded energy-transferring composition without undesirable release.

These nanoparticles are actively taken up by tumor cells, maintaining the two-photon PDT effect in the intracellular environment. The nanoparticles can be used as injectable formulations of two-photon PDT drug, for treatment of deeper tumors with enhanced spatial resolution as well as safe and efficient trafficking to tumor tissues in vivo.

Any photosensitizer which can act as a FRET acceptor with the TPA dyes described herein can be used. An example of a useful photosensitizer for incorporating into the ORMOSIL particles with the TPA dye is 2-devinyl-2-(1-hexyloxyethyl)pyropheophorbide (HPPH). Other suitable photosensitizers include porphines, porphycenes, chlorines, phtalocyanines and the like. The photosensizer should be hydrophobic and should be able to absorb light in the range of the TPA dye aggregate emission spectrum. The ORMOSIL nanoaparticles having incorporated therein a TPA dye and a photosensizer as described herein can be used for PDT applications. The BDSA dye aggregates emits typical orange fluorescence which matches well with the HPPH spectrum for FRET.

To achieve efficient two-photon excitation as well as efficient energy harvesting, the loading density of the energy-donating TPA dye should be higher than that of the energy-accepting photosensitizer. Thus, the ratio of HPPH:TPA dye can be 1:2 to 1:100 by weight percent. It is preferred to have a ratio of 1:50 or less. More preferably, the ratio is between 1:10 to 1:40. In various embodiment, the ratio of HPPH to the TPA dye is 1:5, 1:10, 1:15, 1:20, 1:25, 1:30, 1:35, and 1:40.

The amount of photosensitizer in the nanoparticle should be less than 3 wt %. At higher than 3 wt %, the photosensitizer tends to aggregate which results in fluorescence quenching and reduction in singlet oxygen generation efficiency. In various embodiments, the amount of photosensitizer in the nanoparticle can be 0.1%, 0.5 wt %, 1 wt %, 1.5 wt %, 2 wt % and 2.5 wt %.

In another embodiment, co-incorporation of the TPA dye with the photosensitizer can offer significant advantages for in vitro and in vivo optical imaging and light activated therapy. In particular, organically modified silica nanoparticles can be used to co-entrap a dye and photosensitizer capable of the FRET donor-acceptor pair along with chromophores to increase optical absorptivity of the nanoparticle and fluorescence detection sensitivity due to an increase in Stokes shift. The chromophore molecules, which are not capable of FRET, can be used for an application with double action, i.e., simultaneous PDT tumor treatment and imaging of the tumor. For example, one of the incorporated chromophores can be used for optical imaging, wherein the chromophore is excited in the red-shifted absorption band, while excitation of another molecule with a shorter wavelength can produce phototoxic effect on cells. Suitable chromophores include carbocyanine dye emitting in the near-IR range, Indocyanine Green derivatives and the like.

In addition to the advantages mentioned above, other advantages of the present invention include: simplicity of preparation, minimized release of excess dye, and the ability to surface modify for specific targeting. The following non-restrictive examples are provided to further describe the invention.

### EXAMPLE 1

This example describes synthesis of a TPA dye. 4-(4′-{N-Methyl-N-[2-hydroxyethyl]amino}styryl)styrene (1a). Powder sodium t-butoxide (2.4 g, 25 mmol) was added in small portions to a solution of N-methyl-N-(2-hydroxyethyl)-4-aminobenzaldehyde (3.46 g, 19.3 mmol) and 4-vinylbenzyltriphenylphosphonium chloride^{[14]} (8 g, 19.3 mmol) in methanol (30 mL). The reaction mixture was stirred at room temperature for 1 day and filtered to give a pure trans-isomer precipitate selectively. The filtered product was further washed with methanol several times. Yield 2 g (37%). ¹H NMR (300 MHz, DMSO-d₆): δ 7.68 (d, 2H, J=8.1 Hz), 7.63-7.57 (m, 4H), 7.32 (d, 1H, J=16.4 Hz), 7.12 (d, 1H, J=16.4 Hz), 6.94-6.85 (m, 3H), 6.00 (d, 1H, J=17.4 Hz), 5.41 (d, 1H, J=11.1 Hz), 3.72 (t, 2H, J=5.7 Hz), 3.60 (t, 2H, J=5.7 Hz), 3.14 (s, 3H). 9,10-Bis(4′-{4″-[N-methyl-N-(2-hydroxyethyl)amino]styryl}styryl)anthracene (2). A mixture of 1a (1.2 g, 4.3 mmol), 9,10-dibromoanthracene (0.57 g, 1.7 mmol), Pd(OAc)₂ (26 mg, 0.11 mmol), tri-o-tolylphosphine (88 mg, 0.29 mmol), triethylamine (1 mL), and N-methyl-2-pyrrolidinone (NMP, 6 mL) was introduced into a pressure tube, under argon atmosphere. The reaction mixture was heated for 2 days at 80° C. and poured into methanol after cooling. The filtered precipitate was thoroughly washed with hot ethyl acetate using a Soxhlet apparatus, to give a product (2) pure enough for the next step, as judged by thin layer chromatography eluting with THF/n-hexane (1/1 by volume). Yield 0.54 g (43%). ¹H NMR (300 MHz, DMSO-d₆): δ 8.60 (dd, 4H, J=3.3, 6.6 Hz), 8.32 (d, 2H, J=16.5 Hz), 7.98 (d, 4H, J=8.1 Hz), 7.81 (d, 4H, J=8.1 Hz), 7.77-7.74 (m, 4H), 7.65 (d, 4H, J=8.4 Hz), 7.41 (d, 2H, J=16.2 Hz), 7.21 (d, 2H, J=16.2 Hz), 7.12 (d, 2H, J=16.5 Hz), 6.91 (d, 4H, J=8.4 Hz), 3.75 (t, 4H), 3.62 (t, 4H), 3.17 (s, 6H). 9,10-Bis(4′-{4″-[N-methyl-N-(2-benzoyloxyethyl)amino]styryl}styryl)anthracene (BDSA). To a solution of 2 (0.2 g, 0.27 mmol) in NMP (10 mL) and triethylamine (1 mL), benzoyl chloride (0.2 g, 1.42 mmol) was added slowly in ice bath. After overnight stirring at room temperature, the reaction mixture was poured into brine and extracted with dichloromethane two times. The collected organic phase was dried with MgSO₄ and the solvent was evaporated at reduced pressure. The residue was purified by column chromatography on a silica gel. The eluting impurities were removed using THF/n-hexane (⅓ by volume) and then the product was collected by eluting with dichloromethane. Yield 0.16 g (62%). ¹H NMR (300 MHz, CDCl₃): δ 8.42 (dd, 4H, J=3.3, 6.6 Hz), 7.98 (d, 4H, J=8.1 Hz), 7.94 (d, 2H, J=16.5 Hz), 7.66 (d, 4H, J=8.1 Hz), 7.57 (d, 4H, J=8.1 Hz), 7.54-7.40 (m, 14H), 7.13 (d, 2H, J=16.2 Hz), 6.98 (d, 2H, J=16.2 Hz), 6.94 (d, 2H, J=16.5 Hz), 6.81 (d, 4H, J=8.7 Hz), 4.53 (t, 4H, J=5.7 Hz), 3.80 (t, 4H, J=5.7 Hz), 3.10 (s, 6H). MS (ESI): calcd for C₆₆H₅₇N₂O₄, m/z=941.43 (M+H+); found, m/z=941.3 (M+H+). Anal. Calcd for C₆₆H₅₆N₂O₄: C, 84.23; H, 6.00; N, 2.98; O, 6.80. Found: C, 84.75; H, 6.04; N, 2.85.

### EXAMPLE 2

The ORMOSIL nanoparticles comprising the TPA dye were prepared as follows. N-Methyl-2-pyrrolidinone (NMP, Aldrich) was used as a hydrophilic solvent. To obtain a clear solution of prepolymerized silica sol, 0.2 g of triethoxyvinylsilane (VTES, Aldrich, 97%) in 2 mL NMP was hydrolyzed and condensed in the presence of 40 μL NH₄OH (J. T. Baker, 28.0˜30.0%) at room temperature for 12 h to 1 day, until adding one drop of the resulting solution into excess pure water made white bulk precipitate, without the liquid phase of unreacted VTES or oligomers. After syringe filtering by membrane filter (0.2-μm pore), the sol solution was homogeneously mixed with BDSA or other dyes and additional NMP in a certain ratio. For the study of BDSA optical properties, the mixed solution was prepared such that 6 mg of the total initial feed weight [BDSA+VTES] was dissolved in 0.86 mL of NMP. The compositions (BDSA/[BDSA+VTES]) of 0.5, 5, 25, 50, 75, and 100 wt % were prepared. For other nanoparticles, 0.1 mL of the sol solution was mixed with 0.86 mL of the NMP solution containing a certain amount of BDSA or other dyes, to make a given loading density. The aqueous micelles were prepared by dissolving 0.22 g of Aerosol OT (AOT, sodium bis(2-ethylhexyl)sulfosuccinate, Aldrich, 98%) and 0.4 mL of 1-butanol in 10 mL of deionized water. Nanoprecipitation was induced by one-shot syringe injection of the above mixed NMP solutions (0.72 mL) into the prepared micelle dispersions under vigorous magnetic stirring. The resulting mixtures were further stirred at room temperature, to ensure completion of sol-gel condensation within the co-precipitated nanoparticles. After 1 day of stirring, AOT and 1-butanol were removed by dialyzing the water dispersion against water in a 12˜14 kDa cutoff cellulose membrane for 48 h.

The BDSA composition in the BDSA/ORMOSIL composite nanoparticles (defined as BDSA/[BDSA+VTES] by weight) was varied from 0.5 wt % up to 100 wt %, where the 100 wt % sample is the BDSA-alone nanocrystal. For a quantitative comparison of optical properties depending on the dye loading, the initial feed weights (BDSA+VTES) for the particle preparation were kept constant for samples of all composition. With compositions above 5 wt %, the nanoparticles showed characteristic orange fluorescence of the BDSA aggregate, peaking at above 610 nm (FIG. 4a). Typically, at lower concentrations in the polymer matrix, BDSA emits blue-shifted, greenish fluorescence peaking at ca. 550 nm. The intense and red-shifted orange fluorescence from the obtained nanoparticles is an evidence of the intraparticle aggregation.

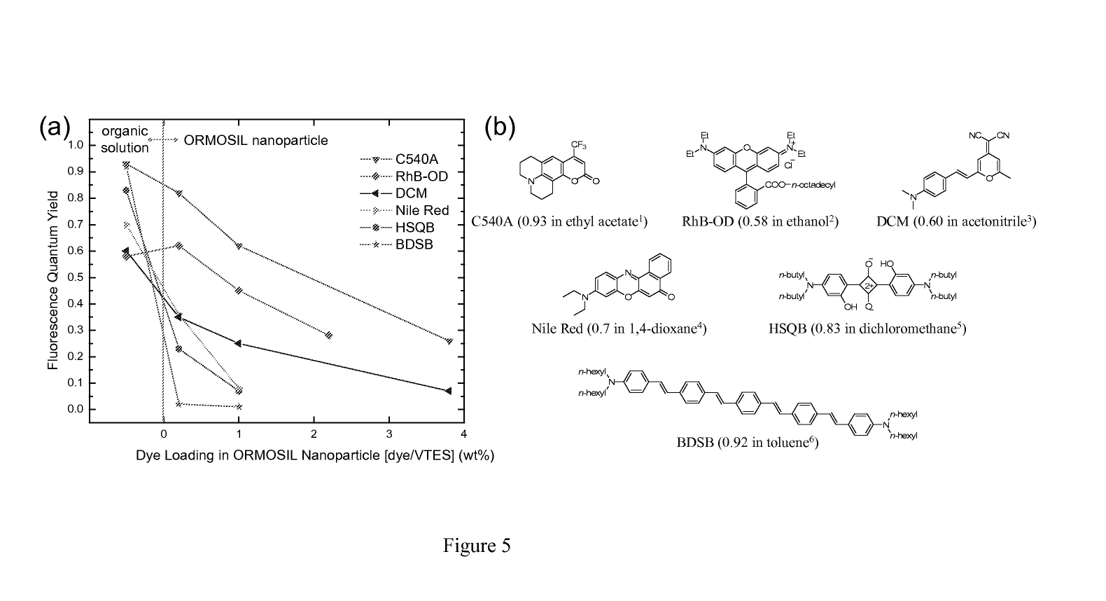

As shown in FIG. 4b, the fluorescence quantum yield does not drop, but rather slightly increases because of increased aggregation. As a result, under the one-photon excitation condition, the total fluorescence output from the same amount of the composite nanoparticles was able to be increased almost linearly by raising the BDSA loading, without any noticeable quenching effect (solid triangle in FIG. 4b). This behavior is directly opposed to concentration quenching of common hydrophobic dyes in ORMOSIL nanoparticles, as examined with coumarin, rhodamine, merocyanine, oxazine, and squaraine derivatives (FIG. 5b) as well as with a typical TPA molecule, where the fluorescence quenching is significant even at dye loadings lower than 5 wt % (see FIG. 5a).

### EXAMPLE 3

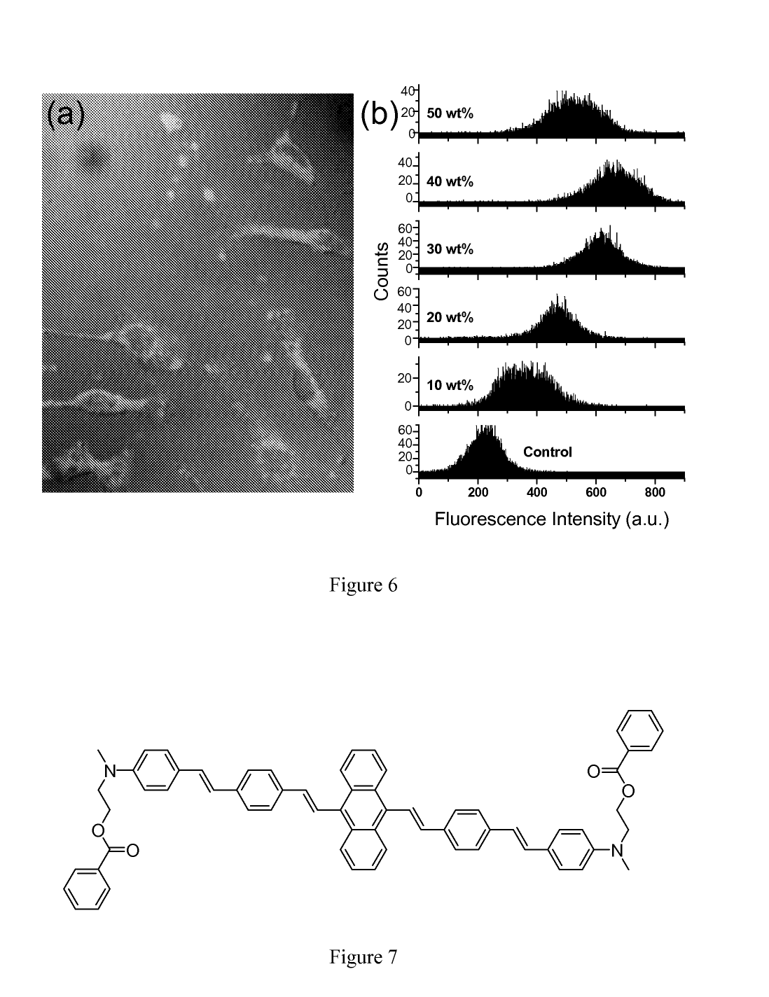

This example demonstrates the use of the BDSA/ORMOSIL nanoparticles for optical bioimaging. For these studies, Human cervical epitheloid carcinoma cell line (HeLa) was maintained in Dulbecco's modified eagle medium with 10% FBS. To study the uptake and imaging of BDSA/ORMOSIL particles, the cells were plated at approximately 10⁵ cells per 35-mm culture plates (glass bottom plates from MatTek Corporation) and 2 mL of the medium was added. For flow cytometry studies, cells were plated at approximately 2.5×10⁵ cells per 25 cm² cell culture flasks and 4 mL media was added. These plates and flasks were then placed in an incubator at 37° C. with 5% CO₂ (VWR Scientific, model 2400). After 24 hrs of incubation, the cells (about 60% confluency) were rinsed with PBS, and fresh media was added. For imaging experiments, 100 μL of the respective nanoparticle sample was added to 1 mL of the cell culture medium and the medium in each plate was exchanged with the nanoparticle mixed medium. For flow cytometry, 200 μL of nanoparticle suspension was mixed with 2 mL of cell culture medium, and medium in each flask was exchanged with this nanoparticle containing medium. Culture plates and flasks were returned to the incubator. After 3 hrs of incubation with nanoparticles, culture plates and flasks were washed thoroughly with PBS to remove free nanoparticles. A fresh medium without serum was added to the 35-mm culture plates for confocal/two-photon imaging, and was directly imaged under a confocal microscope (Leica TCS SP2-AOBS). Confocal images were acquired using 405-nm diode laser excitation, while two photon images were acquired using 800-nm (140 fs pulses at 76 MHz repetition rate) excitation from a Ti: Sapphire laser (Mira from Coherent Inc.) pumped by a 10-W diode pumped solid state laser (Verdi from Coherent Inc.). For flow cytometry measurements, the medium was removed and cells were detached using ethylenediaminetetraacetic acid (EDTA) and trypsin. Cell pellets were collected by centrifugation (500 g, 5 min) and resuspended in PBS. Cell suspensions were analyzed using a FACSCalibur flow cytometer (equipped with 488-nm line from Argon ion laser). Absorption and fluorescence spectroscopy was used to confirm that there is sufficient fluorescence signal from BDSA, under the 488-nm excitation. Data analyses were conducted using a WinMDI program (version 2.8). In spite of the broad size distribution, these composite nanoparticles were avidly internalized into the cell cytoplasm and cells were brightly stained after 3 hrs of incubation, with minimal cytotoxic effect. We also confirmed the minimal cytotoxicity of the nanoparticles using CellTiter-Glo™ luminescent cell viability assay based on the measurement of cellular ATP levels, which showed a minimal decrease in the number of viable cells even after 12 hours of incubation. FIG. 6a shows a representative two photon image of HeLa cells, stained with a composite nanoparticle sample (20 wt % of BDSA loading [BDSA/VTES]). All other composition nanoparticles also showed a similar cellular uptake pattern with reasonable brightness. Though higher loading of BDSA in ORMOSIL particles increased the fluorescence signal from cells, the cell viability was found to be affected above 50 wt % of loading, under visual inspection. The treatment of cells by the composite nanoparticles with 10-50 wt % of BDSA loading, did not show any effect on the cell viability. Further, there was some amount of variability in the cellular uptake from cell to cell, and two-photon fluorescence imaging did not provide enough statistical information on the cellular uptake of the composite nanoparticles with varying BDSA concentration. Therefore, flow cytometry was used to understand the effect of BDSA loading on cellular uptake as well as on the signal output from cells, using the composite nanoparticle samples with 10˜50 wt % of BDSA loading and 488-nm excitation. The signal output from the stained cells was determined to be proportional to both fluorescence intensity and cellular uptake efficiency of individual nanoparticles. The flow cytometry results in FIG. 6b shows that the fluorescence signal from the stained cells increases almost linearly by raising the BDSA loading up to 30 wt %. Above 40 wt % of BDSA loading, intracellular fluorescence does not increase proportional to the increase in fluorescence intensity observed in the extracellular condition (solid triangle in FIG. 4b), indicating that the cellular uptake is hindered at high BDSA loading. Overall, the optimal loading density of BDSA in the BDSA/ORMOSIL composite nanoparticles, in terms of the resulting intracellular signal intensity, can be found at around 30˜40 wt %, which is one or two orders of magnitude higher than the practical values reachable by common organic dyes while preventing concentration quenching. Consequently, with this high number density in the nano-space as well as aggregation-enhanced two-photon absorption and emission, BDSA in the ORMOSIL nanoparticle has the potential to achieve several orders of magnitude improvement in the intracellular two-photon fluorescence signal, over existing fluorescent dyes.

### EXAMPLE 4

This example describes the preparation of ORMOSIL particles comprising BDSA and a photosensitizer, HPPH.

Preparation of Dye-Encapsulating Ormosil Nanoparticles. The nanoparticles, incorporating either one or both of HPPH and BDSA, were synthesized by coprecipitating the dyes with polymeric silica sol in the nonpolar core of AOT/1-butanol micelles in deionized water or D₂O. To obtain a clear solution of organically modified silica sol, 0.2 g of VTES in 2 mL NMP was hydrolyzed and condensed in the presence of 40 μL NH₄OH at room temperature for 12 h˜1 day, until adding one drop of the resulting solution into excess pure water made white bulk precipitate without liquid phase of unreacted VTES or oligomers. After syringe filtering by membrane filter (0.2 μm pore size), 0.15 mL of the sol solution was homogeneously mixed with 0.57 mL NMP solutions containing either or both of HPPH (0.15 mg; 1.1 wt % loading with respect to the added VTES amount) and BDSA (1.35, 2.7, or 4.05 mg; 10, 20, or 30 wt % loading amount, respectively). The micelles were prepared by dissolving 0.22 g of AOT and 0.4 mL of 1-butanol in 10 mL of water or D₂O. Nanoprecipitation was induced by one-shot syringe injection of the above NMP solutions (0.6 mL) into the prepared micelle dispersions under vigorous magnetic stirring. The resulting mixtures were further stirred at room temperature, to ensure completion of sol-gel condensation within the coprecipitated nanoparticles. After 2 days of stirring, AOT and 1-butanol were removed either by dialyzing the water dispersion against water in a 12˜14 kDa cutoff cellulose membrane for 48 h, or by thoroughly washing the D₂O dispersion with n-hexane. Nanoparticles obtained were rigid and spherical.

### EXAMPLE 5

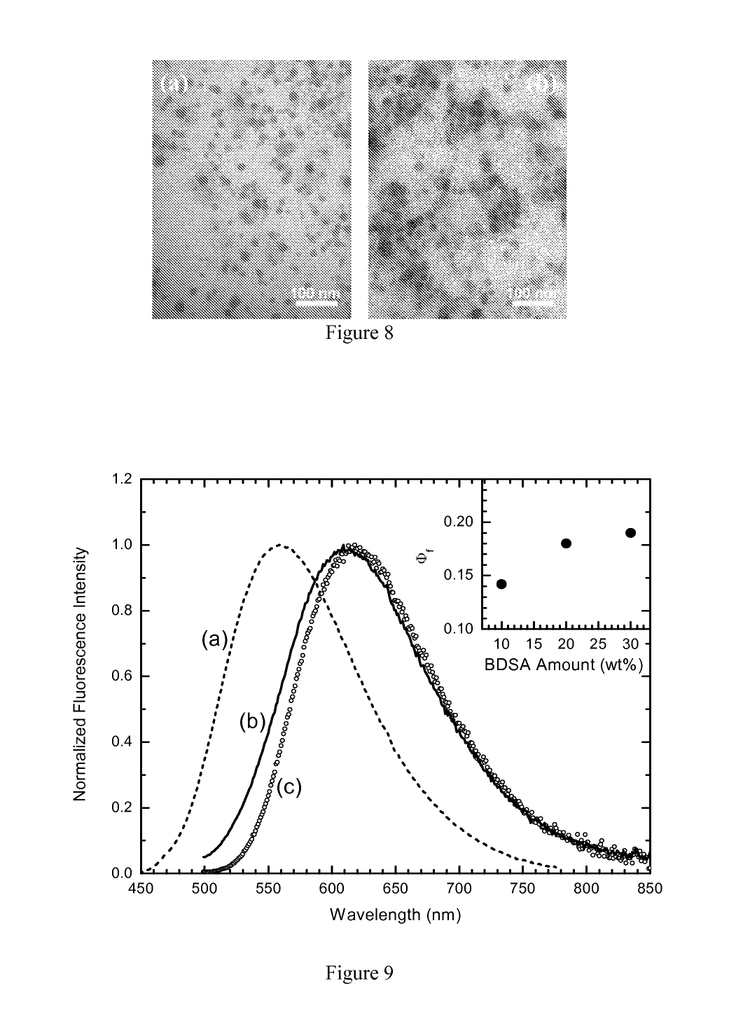

This example describes size characteristics of the particles prepared in Example 4. Transmission electron microscopy (TEM) was performed to determine the size and shape of the prepared nanoparticles, using a JEOL JEM-100cx microscope at an accelerating voltage of 80 kV. FIG. 8 shows representative TEM images of nanoparticles entrapping 1.1 wt % HPPH (a) and 1.1 wt % HPPH/20 wt % BDSA (b), where the sizes are estimated as 15±4 and 22±5 nm, respectively.

### EXAMPLE 6

To produce fluorescent domains of BDSA aggregates in the particle matrix, the following amounts were loaded to induce phase separation: 10, 20, and 30 wt % with respect to the total VTES amount. Fluorescence lifetime measurements were performed using Time correlated Single Photon Counting (TCSPC) technique. For this measurements, Ti:Sapphire laser providing 800 nm femtosecond pulses was used as excitation source and a SPC-830 TCSPC module (Becker & Hickl) with an H7422 (Hamamatsu) detector with a response time in the range of 250 ps was used for the measurements. For measuring the lifetime of donor (BDSA) molecules in presence and absence of acceptor molecules (HPPH), a band pass filter (HQ540/80-2p from Chroma) was used to cut off acceptor emission. For an estimation of the instrument response function (IRF), a dye with a known fluorescence lifetime (from Streak camera measurements), less than 50 ps was used. By deconvoluting this IRF with the obtained lifetime, a lifetime much shorter than the detector response can be estimated. SPCImage software (Becker & Hickl) was used for deconvolution and data fitting for estimation of lifetime values. For quantitative comparison, the concentrations of the nanoparticle water dispersions were kept the same for all the sample sets. The dispersions of all examined HPPH-loaded nanoparticles had the absorbance of 0.07 at 665 nm.

All the prepared nanoparticles incorporating BDSA exhibited almost the same fluorescence spectra peaking at ca. 610 nm. A representative spectrum of the nanoparticles entrapping 20 wt % BDSA is shown in FIG. 9, along with typical state-dependent spectra of BDSA in PMMA films (monomer state at 0.5 wt % and aggregated state at 30 wt %). Typically, at lower concentrations in polymer matrix, BDSA emits blue-shifted, greenish fluorescence, because it exists in the molecularly dispersed monomer state with the limited π-conjugation by a distorted geometry. At the concentrations high enough to induce self-aggregation, the BDSA emission is red-shifted to orange without quenching, owing to its peculiar geometry characteristics. The intense orange fluorescence from the prepared nanoparticles suggests that the loaded BDSA exists in the aggregated state at the studied loading concentrations of 10˜30 wt %. Importantly, as opposed to concentration quenching generally observed for most dye aggregates, fluorescence quantum yields of the BDSA-loaded nanoparticles are rather increased by raising the extent of aggregation with higher loading (the inset of FIG. 9).

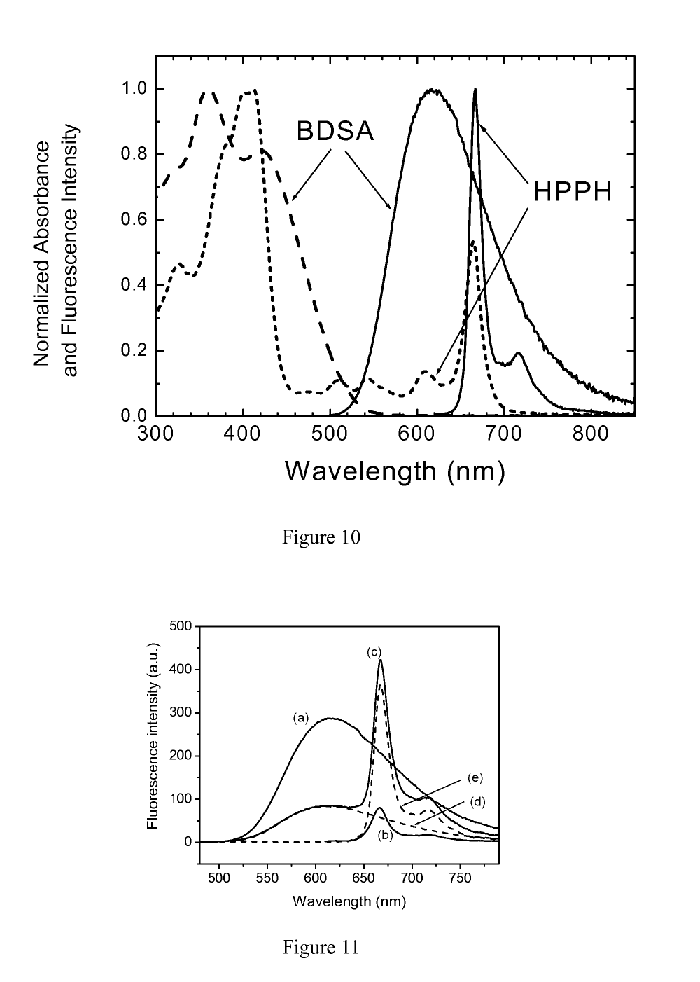

The spectral matching between HPPH and BDSA aggregates, both incorporated into nanoparticles, is shown in FIG. 10. The aqueous dispersion of HPPH-loaded (1.1 wt %) nanoparticles exhibits typical HPPH fluorescence with peak at ˜667 nm, indicating that, by coprecipitation with polymeric VTES sol, the hydrophobic HPPH molecules have successfully been incorporated into the particle matrix without self-aggregation or significant interaction with water. Note that the HPPH fluorescence is completely quenched in water dispersions by self-aggregation. Moreover, the HPPH absorption in nanoparticles has significant spectral overlap with the fluorescence of BDSA aggregates, which enables an energy transfer between them. This spectral matching, together with the aggregation-enhanced fluorescence of BDSA, allow the approach based on the use of BDSA aggregates to overcome the issue of fluorescence quenching in the preparation of donor-concentrated FRET nanoparticles.

FIG. 11 shows a one-photon excited fluorescence spectra of the water-dispersed nanoparticles coencapsulating HPPH (1.1 wt %) and BDSA (20 wt %). When excited at 425 nm, the obtained fluorescence spectrum of the coencapsulating nanoparticles is a composite of the emission contributions from the donor BDSA aggregates and the acceptor HPPH. Compared with the fluorescence intensities of the nanoparticles containing the corresponding amount of each dye, BDSA emission is quenched by 70% and HPPH emission is amplified ca. 5 times, indicating the occurrence of FRET. This energy harvesting evidences that HPPH and BDSA aggregates have successfully been coincorporated into the same nanoparticle by coprecipitation with polymeric VTES sol.

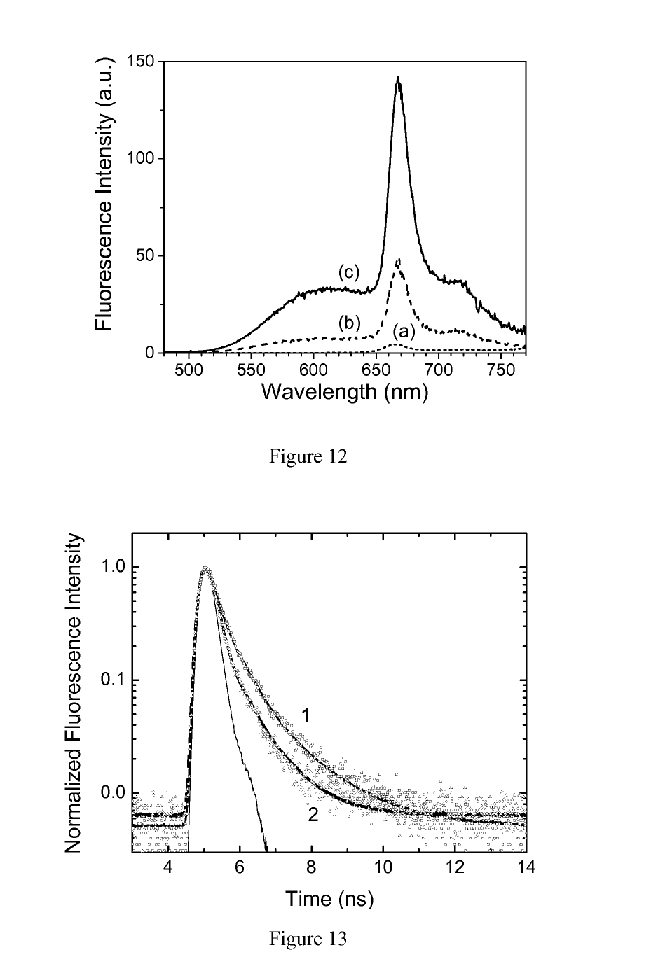

Indirect two-photon excitation of HPPH was examined with the HPPH-loaded nanoparticles with different amounts of BDSA. For quantitative comparison, only the BDSA amount was varied under the same conditions, concentration of the nanoparticles in water dispersion was the same for every sample set. Equality in the amounts of the loaded HPPH was controlled spectrophotometrically, as noted in the Experimental section. FIG. 12 shows the fluorescence spectra of the nanoparticle dispersions, obtained by the excitation at 850 nm. Under this two-photon excitation, the water dispersion of the HPPH-only silica nanoparticles demonstrate weak fluorescence (FIG. 11, (a)). However, the HPPH components from the emission of the HPPH/BDSA coincorporating nanoparticles are amplified by factors of ˜10 and ˜30 for the BDSA loading of 10 and 20 wt %, respectively, which are much greater than the one-photon FRET amplification obtained by the excitation at 425 nm (FIG. 11). More pronounced energy harvesting by two-photon excitation is unambiguously attributed to the large difference in two-photon absorptivities between HPPH and BDSA. Due to the low two-photon absorptivity of HPPH itself, direct excitation of HPPH would have minimal contribution. Therefore, the intense two-photon fluorescence of HPPH is mainly originated from the indirect excitation through the intraparticle FRET, indicating that the energy of the near-IR light is efficiently up-converted by BDSA aggregates to excite HPPH.

To confirm occurrence of FRET, we also used fluorescence lifetime measurements. FIG. 13 shows the fluorescence decay curve with a biexponential fit for nanoparticles doped with (1) 20 wt % BDSA and (2) 1.1 wt % HPPH/20 wt % BDSA. In the case of BDSA incorporated nanoparticles, the fluorescence decay was found to be biexponential in nature with an average lifetime (τₘ) of 636 ps. In case of BDSA and HPPH co-incorporated nanoparticles, the average lifetime of BDSA was found to be around 173 ps, using a biexponential fitting. FRET efficiency of this donor-acceptor pair, can be estimated using the equation, 1-π_{DA}/π_{DA} where π_{DA} is the lifetime of donor in presence of acceptor and π_{D} is lifetime of donor alone. From this, the estimated FRET efficiency of BDSA-HPPH pair when co-doped in silica particles, was found to be ˜73%, which is in close agreement with the FRET efficiency estimated from the fluorescence quenching of donor (BDSA).

### EXAMPLE 7

This example describes the detection of Singlet Oxygen. One- and two-photon induced generations of singlet oxygen were monitored by singlet oxygen luminescence and chemical oxidation methods, respectively. Instead of water, D₂O was used as a dispersion solvent because it extends the lifetime of singlet oxygen. Singlet oxygen luminescence at 1270 nm was recorded for the surfactant-removed D₂O dispersions, using a SPEX 270M spectrometer (Jobin Yvon) equipped with an InGaAs photodetector (Electro-Optical Systems Inc.). A diode-pumped solid-state laser (Millenia, Spectra-Physics) at 532 mn was used as an excitation source. Chemical oxidation of 9,10-anthracenedipropionic acid, disodium salt (ADPA) in the nanoparticle water dispersions was used as a tool to detect singlet oxygen generation. It was monitored by decrease in the absorbance of the added ADPA at 400 nm under excitation with 850 nm. The mixture solutions were prepared by combining 1 mL of the stock dispersions of nanoparticles in water with 0.1 mL of ADPA stock solution in water (0.5 mM). Two-photon excitation at 850 nm was performed by focusing a laser beam through a cuvette (1 cm path length and 2 mm width) containing 0.5 cm³ solution.

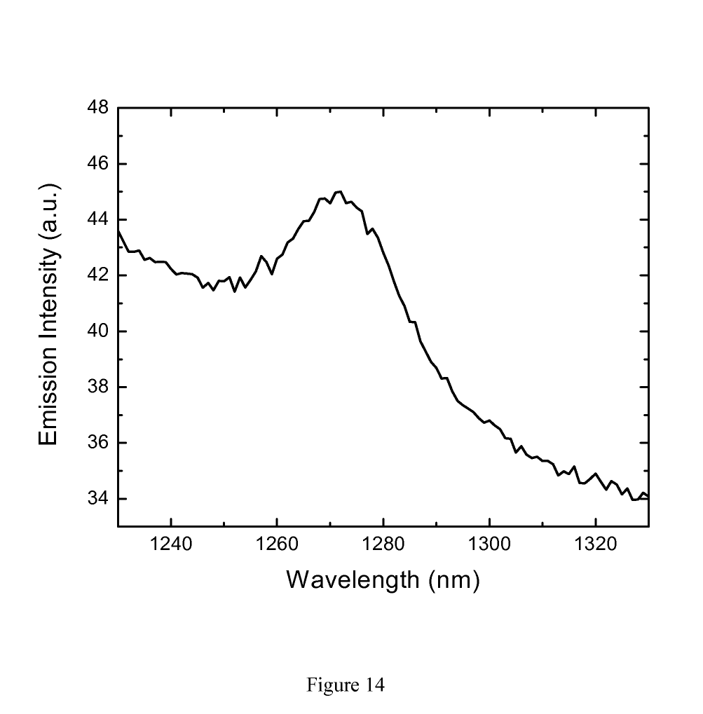

As shown in FIG. 14, the characteristic singlet oxygen emission with peak at 1270 nm was clearly observed under the photoexcitation of the dispersion of nanoparticles coencapsulating HPPH (1.1 wt %) and BDSA (20 wt %), indicating the generation of singlet oxygen (¹O₂) by sensitizing with HPPH.

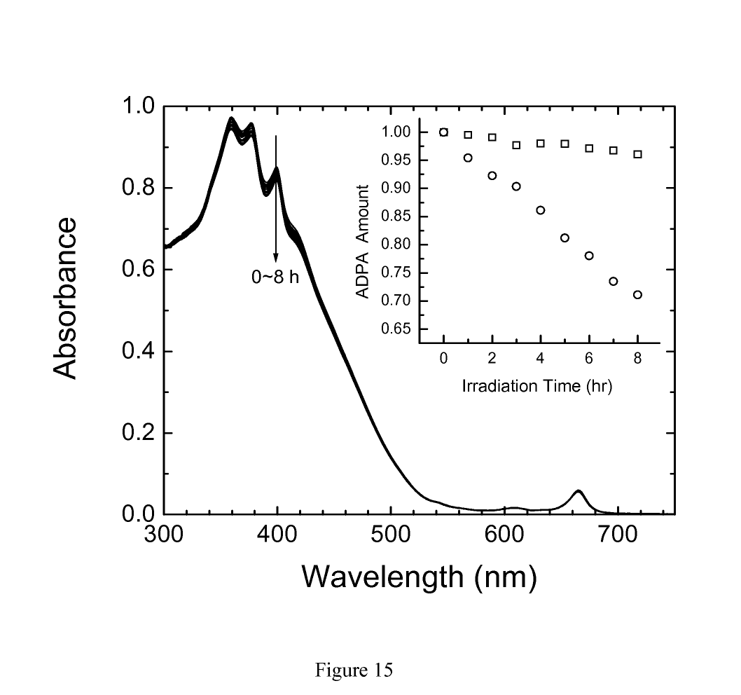

FIG. 15 shows the bleaching of ADPA in water in the presence of nanoparticles coincorporating HPPH (1.1 wt %) and BDSA (20 wt %), under two-photon irradiation at 850 nm, where ADPA has no linear absorption. In the presence of the HPPH/BDSA nanoparticles, the ADPA absorption below ˜450 nm was decreased continuously over the course of irradiation, while HPPH and BDSA showed no noticeable changes in their absorptions above 450 nm, suggesting that both compounds are stable under these irradiation conditions. It should be stressed that the observed photobleaching of ADPA is not caused by its photoreaction in the excited state, but by the oxidation by singlet oxygen liberated from the particles after the generation by two-photon excited photosensitizers entrapped inside. Without coincorporated BDSA aggregates, only a minimal photobleaching of ADPA was observed under the same irradiation conditions, as shown in the inset of FIG. 15. This supports that intraparticle FRET is involved in the two-photon sensitization of singlet oxygen in water.

### EXAMPLE 8

This example describes the uptake of BDSA/HPPH nanoparticles by HeLa cells as described in Example 3. Nanoparticle dispersion was combined with the medium and cells were incubated at 37° C. (5% CO₂) for 3 h. Two-photon laser scanning fluorescence microscopy was performed using a confocal laser scanning microscope (Bio-Rad, model MRC-1024), which was attached onto an upright microscope (Nikon, model Eclipse E800). A water immersion objective lens (Nikon, Fluor-60X, NA 1.0) was used for cell imaging. A long-pass filter (585 LP) was used as emission filters for imaging. For localized spectrofluorometry, the fluorescence signal was collected, without filtering, from the upper port of the confocal microscope, using a multimode optical fiber of core diameter 1 mm, and was delivered to a spectrometer (Holospec from Kaiser Optical Systems, Ann Arbor, Mich.) equipped with a cooled charge coupled device (CCD) camera (Princeton Instruments, Monmouth Junction, N.J.) as a detector.

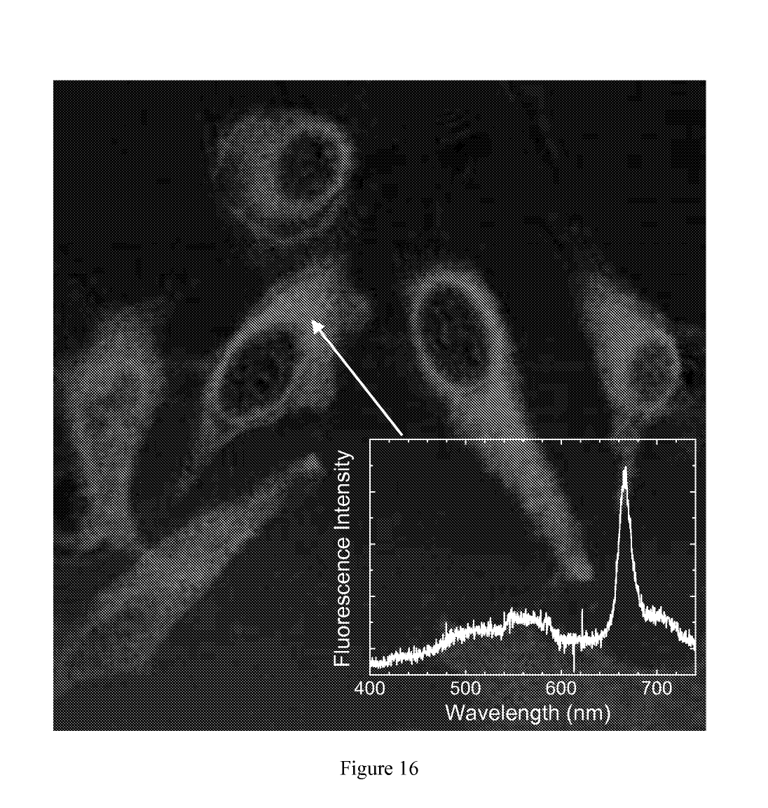

The potential of the HPPH/BDSA coincorporated nanoparticles as a drug-carrier nanoassembly was evaluated in vitro by fluorescence imaging of live tumor cells under two-photon excitation at 850 nm. FIG. 16 shows the two-photon laser scanning microscopic image of the HeLa cells incubated with nanoparticles comprising HPPH (1.1 wt %) and BDSA (20 wt %). An intense fluorescence signal is observed from the cells, indicating active uptake of the nanoparticles by tumor cells. Localized spectrum in the inset of FIG. 10 shows that the two-photon fluorescence from the cytoplasm comprises the characteristic HPPH fluorescence. This affirms that the indirect two-photon excitation of HPPH through intraparticle FRET is still operative in the cellular environment indicating intracellular stability of the ORMOSIL nanoparticles coincorporating HPPH and BDSA.

We claim:

1. Organically modified silica nanoparticles having incorporated therein a two photon absorption (TPA) dye having the formula: 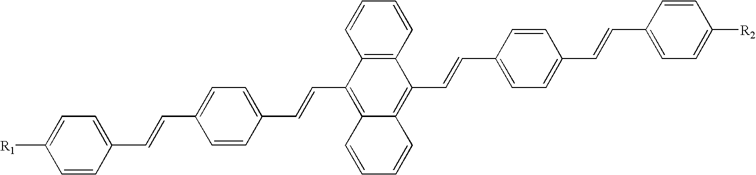
wherein R¹ and R² can independently be —NX¹X², —OX³, or —SX⁴ and X¹, X², X³ and X⁴ can independently be H, alkyl, hydroxyalkyl, benxoyloxyalkyl, phenyl or naphthyl; and a photosensitizer which is activated by fluorescence resonance energy transfer from the TPA dye, and wherein the ratio of the photosensitizer to the TPA dye is between 1:2 to 1:100.

2. The nanoparticles of claim 1, wherein R¹ and R² are the same.

3. The nanoparticles of claim 2 wherein R¹ and R² are N-methyl-N-(2-benzoyloxyethyl.

4. The nanoparticles of claim 1, wherein the photosensitizer is 2-devinyl-2-(1-hexyloxyethyl)pyropheophorbide.

5. The nanoparticles of claim 4 wherein the ratio of the photosensitizer to the TPA dye is 1:20 to 1:30.

6. The nanoparticles of claim 1, wherein the photosensitizer is present from 0.5 wt % to 3 wt %.

7. The nanoparticles of claim 6, wherein the amount of photosensitizer present is between 1 and 2 wt %.

8. The nanoparticles of claim 7 wherein the TPA dye is present from 5 wt % to 50 wt %.

9. The nanoparticles of claim 8, wherein the TPA dye is present from 10-30 wt %.

10. The nanoparticles of claim 1, wherein the average size of the particles is between 10-100 nm.

11. The nanoparticles of claim 10, wherein the average size of the particles is between 15 to 30 nm.

12. The nanoparticles of claim 1, further having incorporated therein a chromophore.

13. Organically modified silica nanoparticles having incorporated therein a TPA dye, said TPA dye having the structure: 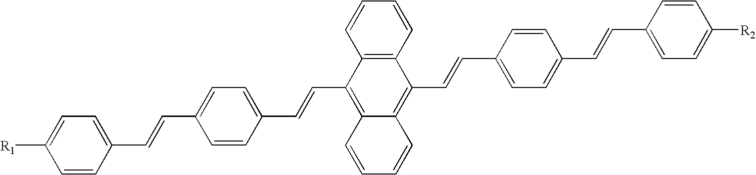
wherein R¹ and R² can independently be —NX¹X², —OX³, or —SX⁴ and X¹, X², X³ and X⁴ can independently be H, alkyl, hydroxyalkyl, benzoyloxyalkyl, phenyl or naphthyl.

14. The nanoparticles of claim 13, wherein R¹ and R² are the same.

15. The nanoparticles of claim 14 wherein R¹ and R² is N-methyl-N-(2-benzoyloxyethyl.

16. The nanoparticle of claim 13, wherein the dye is present between 5 wt % to 50 wt %

17. The nanoparticle of claim 13, wherein the dye is present between 10 wt % to 30 wt %.

18. The nanoparticles of claim 13, wherein the average particle size is between 10 to 100 nm.

19. The nanoparticles of claim 18, wherein the average particle size is between 15 to 30 nm.

20. A composition comprising the nanoparticles of claim 13.
